(19) United States
(12) Patent Application Publication
(10) Pub. No.: US 2006/0205007 A1
(43) Pub. Date: Sep. 14, 2006

(54) DNA supporting fiber and DNA supporting fiber sheet and methods of producing them

(75) Inventors: Zuyi Zhang, Yokohama-shi (JP); Toshiya Yuasa, Kawasaki-shi (JP); Shinji Eritate, Kawasaki-shi (JP); Yoshinori Kotani, Yokohama-shi (JP); Masaaki Kawabe, Kitasaitama-gun (JP); Tatsuo Nakamura, Oura-gun (JP); Koichi Kato, Koga-shi (JP); Yoshiyuki Tozawa, Kitasaitama-gun (JP)

Correspondence Address: FITZPATRICK CELLA HARPER & SCINTO, 30 ROCKEFELLER PLAZA, NEW YORK, NY, 10112, US

(73) Assignee: Canon Kabushiki Kaisha, Tokyo (JP)

(21) Appl. No.: 11/436,598

(22) Filed: May 19, 2006

Related U.S. Application Data
(63) Continuation of application No. PCT/JP05/21623, filed on Nov. 18, 2005.

(30) Foreign Application Priority Data
Nov. 26, 2004 (JP) 2004-342888

(51) Int. Cl. C12Q 1/68; G01N 1/28; C12M 1/34; B05D 3/02

(52) U.S. Cl. 435/6; 435/287.2; 427/2.11

(57) ABSTRACT

There is provided a DNA supporting fiber capable of maintaining the stability of DNA and efficiently expressing the adsorption property of DNA. Also provided is a DNA supporting sheet useful in a variety of applications, the sheet that utilizes the fiber. The DNA supporting fiber is produced by fusing and fixing, onto the surface composed of a thermoplastic resin of a fiber, particles where DNA as an adsorbent is immobilized in a porous matrix containing an inorganic oxide.

This application is a continuation of International Application No. PCT/JP2005/021623 filed on Nov. 18, 2005, which claims the benefit of Japanese Patent Application No. 2004-342888 filed on Nov. 26, 2004.

### BACKGROUND OF THE INVENTION

1. Field of the Invention

The present invention relates to a DNA supporting fiber that is useful in environmental cleanup by way of the adsorption and elimination of mutagens for eliminating, from environment, mutagens that act on the genes of organisms and cause mutation, and is also useful in substance separation for selectively separating a variety of substances. The present invention also relates to a method of producing the DNA supporting fiber, and to a sheet comprising the DNA supporting fiber.

2. Related Background Art

As studies on the replication of biological individuals move forward, the subjects of the studies went beyond understanding a vital activity and are now directed to the use of genes that play a central part in this activity, particularly genes that exhibit a variety of functions ex vivo (hereinafter, simply referred to as DNA (deoxyribonucleic acid)).

By way of example, Japanese Patent Application Laid-Open No. H10-175994 (Patent Document 1) discloses a technique for immobilizing DNA on a variety of immobilizing carriers. According to this disclosed technique, the immobilizing carriers are composed of an inorganic solid material and can be shaped in the form of a powder, a bulk, a film, a plate, a tube, a fiber, an assembly thereof, a porous material composed of them, and the like. As described therein, the composition of the immobilizing carriers includes oxides, complex oxides, carbides, halides, nitrate, phosphate and sulfate. To be more specific, a wide range of forms such as phosphate and calcium salt such as hydroxyapatite, silica gel and other silicates, glass wool, rock wool and woven and nonwoven cloth thereof can be applied to the composition of the immobilizing carriers. DNA immobilized in such a form is not limited to DNA used alone and is exemplified by DNA immobilized together with a polysaccharide, a derivative thereof or a protein such as collagen, and DNA immobilized as a complex with alginic acid. This Patent Document 1 describes the examination of DNA immobilized composites constructed in various forms for the elution rate of DNA immobilized therein as well as results of evaluating the DNA immobilized composites for their activities in adsorbing ethidium bromide as a mutagen.

Alternatively, Japanese Patent Application Laid-Open No. 2001-081098 (Patent Document 2) discloses a water-insoluble DNA cross-linked product and a method of using the water-insoluble DNA cross-linked product as an environmental cleanup material. This water-insoluble DNA cross-linked product has been achieved by cross-linking double-stranded DNAs using

UV irradiation under conditions where the double-stranded DNAs are in the water or free from solvents. After an aqueous solution of water-soluble DNA or the like is used to coat a support forming a layer of the solution or a thin film, DNA is self-cross-linked and in solubilized by UV irradiation. DNA that is preferably used in this technique is exemplified by those derived from the testes of fishes or the thymus glands of animals and concretely exemplified by DNA from salmon, herring and cod soft roes (testes) or synthetic DNA having a poly(dA)-poly(dT)-type sequence. The shape and material of such a support include a plate, a sphere (e.g., a sphere having a diameter of 0.1 mm or 10 mm) or a fiber, which may have a porous structure. Other examples thereof disclosed therein include such as synthetic resins, glasses, ceramics, metals or natural fibers (e.g., cellulose or pulp as well as chemically processed products thereof). Such a cross-linked product is useful in applications such as filter media (e.g., cigarette filters, gas filter media of air cleaners, and liquid filter media of drinking water, edible water, beverages and foods), adsorbents and environmental clean up materials for immobilizing environmental hormone and toxic metals.

On the other hand, Japanese Patent Application Laid-Open No. 2004-003070 (Patent Document 3) discloses a fiber or a fiber sheet having at least a surface comprising a thermoplastic resin and carrying solid particles affixed to the surface and a process for manufacturing the fiber or the fiber sheet. When compared to conventional techniques that immobilize solid particles into a fiber with a binder or the like, a technique described in this document can provide a fiber or a fiber sheet where solid particles are uniformly bonded onto the surface of the fiber, with their surface properties effectively retained.

### SUMMARY OF THE INVENTION

The present inventors have suggested a DNA immobilized material as a material that is capable of promoting a wide range of applications such as the adsorption and elimination of mutagens and the like and substance separation. Such a DNA immobilized material can be applied to filter media and the like by a method in which a fiber or a fiber sheet shaped in advance in sheet form is directly coated with a dispersion solution containing DNA so that the DNA is bonded and supported on the fiber or the fiber sheet. This method that uses the dispersion solution might present problems such as a limitation on the amount of DNA supported on the DNA immobilized material and a blockage in pores between fibers. When a method, in which a DNA material is directly embedded into a thermoplastic fiber, is employed, the DNA immobilized material is exposed to high temperatures for a long time during kneading into the fiber and melt spinning. Therefore, in many cases, the method presents a problem with the inevitable deterioration of the function of DNA. Thus, under present circumstances, there is no effective solution to the problem associated with the immobilization of substances having low thermal stability such as DNA in techniques for fusing DNA to a fiber having a surface composed of a thermoplastic resin.

Under the circumstances, there has been a strong demand for the development of a DNA supporting fiber suitable for fiber media, which reduces the deterioration of the stability of DNA and expresses the function of DNA with high efficiency. Thus, an object of the present invention is to provide a DNA supporting fiber capable of maintaining the stability of DNA and efficiently expressing the adsorption property of DNA and to provide a DNA supporting sheet useful in a variety of applications that utilize the DNA supporting fiber.

For attaining the above-described object, a DNA supporting fiber according to a first invention of the present application is a DNA supporting fiber having a surface to which DNA immobilized particles are bonded, characterized in that the DNA immobilized particles are particles where DNA is immobilized in a porous matrix.

A DNA supporting fiber sheet according to a second invention of the present application is characterized in that the DNA supporting fiber according to the first invention is shaped into a sheet as a fiber assembly.

In addition, a method of producing a DNA supporting fiber according to a third invention of the present application is a method of producing a DNA supporting fiber having a surface to which DNA immobilized particles are bonded, characterized by comprising the step of heat sealing DNA immobilized particles where DNA is immobilized in a porous matrix to the surface including a thermoplastic resin of a fiber by supplying the DNA immobilized particles to the surface of the fiber under heating.

According to the invention of the present application, the use of the DNA immobilized particles where DNA is immobilized in a porous matrix markedly improves the stability of DNA against heat and the like and allows the easy and firm immobilization of DNA on the surface of a fiber without deteriorating the function of DNA. The DNA supporting fiber thus obtained can be utilized as a fiber material for fabrics, nonwoven cloth, and the like. For example, cloth, a fiber bundle, a sheet or nonwoven cloth that uses this DNA supporting fiber can be utilized as a fiber medium, an adsorbent, and so on, which can markedly improve contact efficiency with gas or liquid and can sufficiently exhibit adsorption function originating from DNA. Furthermore, the present invention favorably works as a filter, which can greatly reduce the elution of DNA when used in the water and is less likely to undergo the decomposition of DNA by microorganisms or the like, because the DNA is confined in the porous matrix.

Other features and advantages of the present invention will be apparent from the following description taken in conjunction, in which like reference characters designate the same or similar parts throughout the figures thereof.

### DESCRIPTION OF THE PREFERRED EMBODIMENTS EMBODIMENT(S)

The present invention provides a DNA supporting fiber having a surface to which DNA immobilized particles are bonded, a DNA supporting fiber sheet comprising this DNA supporting fiber, a DNA supporting filter composed of the DNA supporting fiber sheet, and a method of producing the DNA supporting fiber. The “DNA immobilized particles” used in the present invention refer to solid particles where DNA is immobilized in a porous matrix. The immobilized DNA maintains adsorption function intended by the present invention. The porous matrix is a wall portion that divides a large number of fine pores and assumes the form of, for example, a mesh structure that contains voids serving as the fine pores and a fine pore wall that divides the fine pores. The structure of this porous matrix can be observed with FE-SEM. “Bonded” or “bonding” used herein means that the particles are tightly attached to the surface of the fiber without falling off the surface due to a flow of gas or water.

The present inventors have made the patent applications on the inventions relating to: an immobilized DNA obtained from a dispersion solution containing an oxide colloid and DNA with them dispersed for preventing the elution of DNA in the water and maintaining its stability; and a technique for immobilizing DNA, which uses a DNA immobilized porous oxide gel obtained by removing a dispersion medium from a dispersion solution containing an oxide colloid, basic functional siloxane and DNA with them dispersed (Japanese Patent Application Laid-Open Nos. 2003-152619 and 2004-207253). DNA composites obtained by these techniques are provided with fine pores necessary for the infiltration of gas and liquid and can be utilized as an excellent environmental filter medium.

The DNA immobilized particles have a structure where DNA is immobilized in a porous matrix. The immobilization of DNA in a porous matrix alleviates the deterioration of DNA caused by heat during the process of bonding the DNA onto a fiber and reduces the deterioration of the adsorption property of the DNA that has been bonded on the fiber. Such a porous matrix can appropriately be selected from the group consisting of metals, polymers, metal halide compounds, oxides and complexes thereof. This matrix can be formed by any means selected preferably from means in which a dispersion solution containing DNA and components of the matrix with them dispersed is directly solidified, and means in which a dispersion solution of DNA is immersed in the porous matrix formed in advance and then solidified. However, the matrix must have a porous structure where DNA is immobilized in a large number of fine pores that are left open to the outside of the DNA immobilized particle. Preferably, the porous matrix contains an inorganic oxide from the viewpoint of being capable of attaining heat resistance and contact with the outside through the fine pores as described above. A porous matrix mainly composed of an inorganic oxide is more preferred because heat resistance and DNA immobilizing function originating from the inorganic oxide can effectively work.

DNA immobilized particles of a porous inorganic oxide obtained by gelation of an inorganic oxide from a colloidal solution containing a colloid of the inorganic oxide and DNA with them dispersed (hereinafter, referred to as DNA immobilized gel particles) can preferably be utilized as the DNA immobilized particles where the porous matrix is mainly composed of the inorganic oxide. This gelation can be performed by, for example, a method that allows the secondary flocculation of this colloid of the inorganic oxide in the process of removing a dispersion medium from the colloidal solution. This secondary flocculation can also be brought about by addition of an ion or a solvent that causes the secondary flocculation. The resulting gel is finally dried and can be used as the DNA immobilized gel particles to be bonded onto the fiber. Examples of the colloid of the inorganic oxide can include colloidal silica, colloidal aluminum oxide, colloidal iron oxide, colloidal gallium oxide, colloidal lanthanum oxide, colloidal titanium oxide, colloidal cerium oxide, colloidal zirconium oxide, colloidal tin oxide and colloidal hafnium oxide. In light of the stability of the dried gel and cost performance, it is preferred to use at least colloidal silica.

When DNA is immobilized using a mixture of the colloid of the inorganic oxide containing or mainly composed of colloidal silica, it is more preferred to adopt a preparation obtained by supplementing colloidal silica as a main component with a colloid of one or two or more metal oxide(s) containing a trivalent or tetravalent metal which can be selected from the group consisting of aluminum oxide, iron oxide, titanium oxide and zirconium oxide. The addition of a colloid of metal having the number of valence of three (trivalent metal) or four (tetravalent metal) forms the binding between the phosphate functional moiety of DNA and the metal ion. As a result, DNA in a gel state can be supported more firmly in the oxide gel and is inhibited from falling off the gel, for example, in the water. The content of the trivalent or tetravalent metal oxide with respect to the total amount of the colloidal silica and the trivalent or tetravalent inorganic oxide is preferably 0.1 to 50% by weight in terms of solid content of the colloid. Any of these colloids can be synthesized by hydrothermal reaction, and some of them are commercially available in the form of aqueous colloidal dispersions. The ratio of DNA/inorganic oxide is 0.1/99.9 to 25/75 by weight, more preferably 0.5/99.5 to 10/90 by weight, in terms of solid contents. The dispersion solution of the colloid thus obtained is conjugated with a DNA aqueous solution. A dispersion medium is then removed by a method such as heating, spray drying or vacuum drying to form a gel of the DNA conjugated oxide. This yields, as a secondary flock, DNA immobilized gel particles available in the present invention. For enhancing gel strength, it is preferred that heating treatment should be applied to the gel to the extent that does not cause the decomposition of DNA. A temperature not higher than 200° C., more preferably not higher than 150° C., at which the effect of enhancing gel strength can be obtained by heating, is adopted as the heating temperature. A third component may be added, if necessary, for the purpose of strengthening the binding between colloids of an inorganic oxide through secondary flocculation and preventing flocculation between DNA and the colloids and the flocculation of the colloids in the dispersion solution. This third component can include, but not particularly limited to, suitable additives such as acids, bases, water-soluble metal compounds and metal alkoxide, which promote the flocculation of the colloids.

Moreover, a polymer with a basic functional moiety can preferably be used as an auxiliary component in the porous matrix containing colloidal silica. In this case, the basic functional moiety forms an acid-base structure with a phosphate moiety of DNA to thereby allow the firm immobilization of DNA in the porous matrix, with its double helix maintained. A preferred basic polymer is polyorganosiloxane with a basic functional moiety. Preferably, the polyorganosiloxane with a basic functional moiety is any of those facilitating the preparation of a uniform dispersion/dissolution solution of colloid particles and DNA when the DNA immobilized porous oxide is produced. Such polyorganosiloxane with a basic functional moiety can be obtained by hydrolyzing and condensing a silane compound with a basic functional moiety. Preferred concrete examples of the silane compound with a basic functional moiety can include any one or two or more of compounds represented by the formulas (1) to (5).

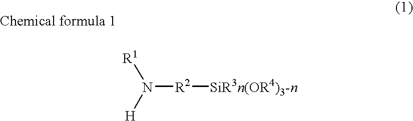

In the formula (1), R¹ is selected from the group consisting of hydrogen or a monovalent carbon hydride moiety having 1 to 8 carbon atoms; R³ and R⁴ each independently represent a monovalent carbon hydride moiety having 1 to 8 carbon atoms; R² is selected from the group consisting of a divalent carbon hydride moiety having 1 to 8 carbon atoms and a divalent moiety having —NH—; and n is selected from the group consisting of 0, 1 and 2.

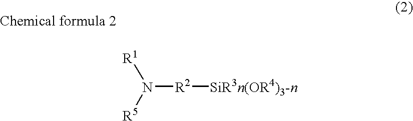

In the formula (2), R¹, R³, R⁴ and R⁵ each independently represent a monovalent carbon hydride moiety having 1 to 8 carbon atoms; R² is selected from the group consisting of a divalent carbon hydride moiety having 1 to 8 carbon atoms and a divalent moiety having —NH—; and n is selected from the group consisting of 0, 1 and 2.

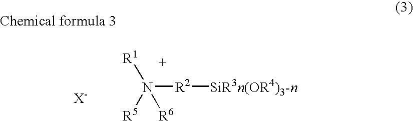

In the formula (3), R¹, R³, R⁴, R⁵ and R⁶ each independently represent a monovalent carbon hydride moiety having 1 to 8 carbon atoms; R² is selected from the group consisting of a divalent carbon hydride moiety having 1 to 8 carbon atoms and a divalent moiety having —NH—; n is selected from the group consisting of 0, 1 and 2; and X⁻ represents an anion.

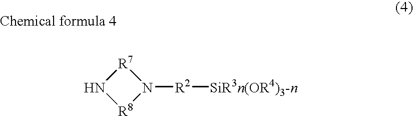

In the formula (4), R³ and R⁴ each independently represent a monovalent carbon hydride moiety having 1 to 8 carbon atoms; R⁷ and R⁸ each independently represent a divalent carbon hydride moiety; R² is selected from the group consisting of a divalent carbon hydride moiety having 1 to 8 carbon atoms or a divalent moiety having —NH—; and n is selected from the group consisting of 0, 1 and 2.

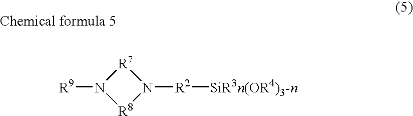

In the formula (5), R³, R⁴ and R⁹ each independently represent a monovalent carbon hydride moiety having 1 to 8 carbon atoms; R⁷ and R⁸ each independently represent a divalent carbon hydride moiety; R² is selected from the group consisting of a divalent carbon hydride moiety having 1 to 8 carbon atoms and a divalent moiety having —NH—; and n is selected from the group consisting of 0, 1 and 2.

Examples of the monovalent carbon hydride moiety having 1 to 8 carbon atoms represented by R¹, R³, R⁴, R⁵, R⁶ or R⁹ in these formulas (1) to (5) can include a chain, branched or cyclic alkyl moiety having 1 to 8 carbon atoms such as methyl, ethyl, n-propyl, s-propyl, n-butyl, s-butyl, n-pentyl, n-hexyl, n-heptyl and n-octyl moieties and an aromatic carbon hydride moiety such as a phenyl moiety. The divalent carbon hydride moiety having 1 to 8 carbon atoms represented by R² in the formulas (1) to (5) can include a chain, branched or cyclic divalent alkylene moiety having 1 to 8 carbon atoms such as methylene, ethylene, trimethylene and tetramethylene moieties and a divalent aromatic carbon hydride moiety having 1 to 8 carbon atoms such as o-phenylene, m-phenylene and p-phenylene moieties. The divalent moiety having —NH— represented by R² in the formulas (1) to (5) can specifically include a —NH— moiety and a moiety formed by the binding of one or two of divalent carbon hydride moieties such as methylene, ethylene, trimethylene and tetramethylene moieties to a nitrogen atom, which can concretely exemplified by —C₂H₄NHC₃H₆—, —C₃H₆NHC₂H₄—, —CH₂NHC₃H₆—, —C₂H₄NHCH₂—, —C₂H₄NHC₂H₄— and —C₃H₆NHC₃H₆— (the alkylene moiety of these moieties may be linear or branched). The divalent carbon hydride moiety represented by R⁷ or R⁸ in the formulas (4) to (5) is not limited by the number of a carbon atom and can include a chain, branched or cyclic divalent alkylene moiety such as methylene, ethylene, trimethylene and tetramethylene moieties and a divalent aromatic carbon hydride moiety such as o-phenylene, m-phenylene and p-phenylene moieties. To be more specific, it can be exemplified by methylene and ethylene moieties. The anion represented by X⁻ in the formula (3) may be any of those capable of forming an ion pair with the cation of siloxane having a quaternary amino moiety and can include a halogen ion.

The compounds represented by the formulas (1) to (3) can concretely include H₂NC₃H₆Si(OCH₃)₃, H₂NC₃H₆SiCH₃(OCH₃)₂(CH₃)HNC₃H₆Si(OCH₃)₃, (CH₃)HNC₃H₆SiCH₃(OCH₃)₂, (CH₃)HNC₃H₆Si(OC₂H₅)₃, (CH₃)HNC₃H₆SiCH₃(OC₂H₅)₂, (CH₃)₂NC₃H₆Si(OCH₃)₃, (CH₃)₂NC₃H₆SiCH₃(OCH₃)₂, (CH₃)₂NC₃H₆Si(OC₂H₅)₃, (CH₃)₂NC₃H₆SiCH₃(OC₂H₅)₂, (C₂H₅)₂NC₃H₆Si(OCH₃)₃, (C₂H₅)₂NC₃H₆Si(OC₂H₅)₃, H₂NC₂H₄NHC₃H₆Si (OCH₃)₃, (CH₃)HNC₂H₄NHC₃H₆Si(OCH₃)₃, H₂NC₂H₄NHC₃H₆SiCH₃(OCH₃)₂, (CH₃)HNC₂H₄NHC₃H₆SiCH₃(OCH₃)₂, H₂NC₂H₄NHC₃H₆Si(OC₂H₅)₃, (CH₃)HNC₂H₄NHC₃H₆Si(OC₂H₅)₃, CH₃HNC₂H₄NHC₃H₆SiCH₃(OC₂H₅)₂, (CH₃)₂NC₂H₄NHC₃H₆Si(OCH₃)₃, (CH₃)₂NC₂H₄NHC₃H₆SiCH₃(OCH₃)₂, (CH₃)₂NC₂H₄NHC₃H₆Si(OC₂H₅)₃, (CH₃)₂NC₂H₄NHC₃H₆SiCH₃(OC₂H₅)₂, Cl⁻(CH₃)₃N⁺C₃H₆Si(OCH₃)₃, Cl⁻(C₄H₉)₃N⁺C₃H₆Si(OCH₃)₃ (the alkyl and alkylene moieties of these compounds may be linear or branched).

The compounds represented by the formulas (4) and (5) can concretely include compounds represented by the formulas (4) and (5) in which R², R⁷ and R⁸ each represent, for example, a divalent carbon hydride moiety such as methylene, ethylene and trimethylene moieties and R³, R⁴ and R⁹ each represent a monovalent carbon hydride moiety such as methyl, ethyl and propyl moieties. Especially preferred examples thereof can include a compound represented by the formula (6).

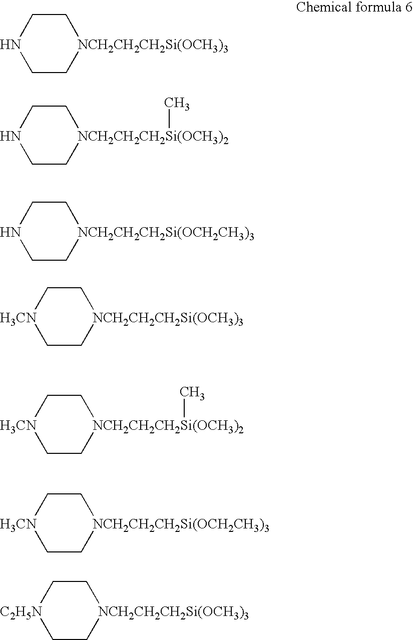

Among these basic functional moieties, basic functional moieties containing secondary, tertiary and quaternary amino moieties are especially preferred. The polyorganosiloxane with a basic functional moiety preferably applied to the third component of the present invention can be obtained as a hydrolysis condensate of a siloxane compound with a basic functional moiety by dispersing or dissolving a silane compound with a basic functional moiety in an aqueous dispersion medium or solvent. The silane compound with a basic functional moiety that is preferably used in the present invention is any one or two or more of the silane compounds with a basic functional moiety represented by the formulas (1) to (6). This polyorganosiloxane may optionally be any of those containing an alkylsiloxane component or/and a phenylsiloxane component within a range that does not impair the object and effect of the present invention. As an example, the polyorganosiloxane with a basic functional moiety that contains such a component may be a copolymer obtained by adding, for example, an alkylsilane compound or/and a phenylsilane compound to the above-described silane compound with a basic functional moiety, which is in turn subjected to hydrolysis and condensation polymerization.

For hydrolyzing a silane compound with a basic functional moiety to form polyorganosiloxane with a basic functional moiety, the silane compound with a basic functional moiety may directly be added to water and then hydrolyzed; or otherwise, the silane compound with a basic functional moiety may be hydrolyzed after being supplemented with an organic dispersion medium such as alcohol or ketone and subsequently with water or after being added to the mixed dispersion medium of an organic dispersion medium such as alcohol or ketone with water. Any of those containing an organic dispersion medium may be subjected to solvent replacement by water, if necessary, to obtain an aqueous dispersion solution of siloxane with a basic functional moiety.

When polyorganosiloxane with a basic functional moiety is used in the porous matrix, the ratio of the polyorganosiloxane with a basic functional moiety/the inorganic oxide that forms a colloid is preferably 0.1/99.9 to 25/75 by weight, more preferably 0.5/99.5 to 10/90 by weight. If the ratio of the polyorganosiloxane with a basic functional moiety/the inorganic oxide is 0.1/99.9 or more by weight, DNA is appropriately immobilized in the porous matrix through the binding between the phosphate moiety of the DNA and the basic functional moiety of the polyorganosiloxane. The ratio of 0.5/99.5 or more by weight produces this effect more remarkably. On the other hand, if the ratio of the polyorganosiloxane with a basic functional moiety/the inorganic oxide is 25/75 or less by weight, fine pores are efficiently formed between colloids of the oxide. The ratio of 10/90 or less by weight produces this effect more remarkably. The ratio of the DNA/the oxide matrix is preferably 0.1/99.9 to 25/75 by weight, more preferably 0.5/99.5 to 10/90.

As described above, the fine pores formed in the porous matrix have the function of immobilizing DNA therein and the function as a site that allows the contact of DNA with a substance captured by the DNA. The colloid of the inorganic oxide that is capable of forming such fine pores has a diameter of preferably 5 to 100 nm, more preferably 10 to 50 nm. If the colloid of the inorganic oxide has a diameter of 5 nm or more, the size of a fine pore is kept large and DNA comes into sufficient contact with a substance to be captured by the DNA. The colloid of the inorganic oxide having a diameter of 10 nm or more produces this effect more remarkably. On the other hand, if the colloid of the inorganic oxide has a diameter of 100 nm or less, a large number of fine pores can be secured while DNA is inhibited from being eluted into an aqueous solution and is therefore firmly immobilized in the porous matrix. The colloid of the inorganic oxide having a diameter of 50 nm or less produces this effect more remarkably.

The DNA immobilized gel particles thus obtained are provided as particles having varying particle sizes in which colloids having diameters in the above-described range are flocculated. However, for immobilizing the particles in the DNA supporting fiber and the DNA supporting fiber sheet as described below, it is preferred that the particle sizes of the particles should be rendered uniform within a fixed range. In order to achieve the particle sizes rendered uniform within a fixed range, a spray drying method can be used in the process of obtaining a dried gel as described above. When the dried gel is prepared as a bulk product, the gel can be utilized after being pulverized by a well known apparatus, for example, a mill. The DNA immobilized gel particles suitable in the present invention have a particle size of 0.1 μm to 500 μm, more preferably 1 μm to 100 μm.

Next, means for bonding the DNA immobilized particles onto the fiber or the fiber sheet will be described. A technique for bonding the DNA immobilized particles is not particularly limited as long as the use of the technique allows the immobilization of the DNA immobilized particles onto the surface of the fiber. When the above-described DNA immobilized gel particles are used as the DNA immobilized particles, for example, the technique described in the above Patent Document 3 can preferably be utilized. That is, a fusion apparatus based on this technique has preliminary heat means for maintaining the DNA immobilized gel particles at a fixed temperature and particle contact means for bonding the heated particles to the fiber or the fiber sheet. A fiber having at least a partial or entire surface composed of a thermoplastic resin is used as a fiber material. The thermoplastic resin in the surface of the fiber that is used in the present invention includes, but not particularly limited to, a thermoplastic resin that allows the fiber to have at least a surface whose melting point is 200° C. or lower, preferably 170° C. or lower, more preferably 150° C. or lower, in light of the heat stability of DNA. If the melting point is higher than 200° C., the temperature of the DNA immobilized gel particles and/or the temperature of an air stream for leading the particles to collide with the surface of the fiber must be set to a temperature higher than 200° C. Therefore, reduction in the adsorption property caused by the deterioration of DNA might be more likely to occur. Thus, it is preferred to adopt a thermoplastic resin composing the surface of the fiber that has a melting point of a relatively low temperature at which the DNA immobilized gel particles can be bonded onto the fiber, and to adopt means for alleviating thermal influence on DNA in the way that the DNA immobilized gel particles are subjected to preliminary heat and then transferred to the surface of the fiber or the fiber sheet via an air stream at a relatively high temperature. In the later case, a lower limit on the melting point of the thermoplastic resin composing the surface of the fiber is not particularly restricted. However, a material having an exceedingly low melting point such as paraffin lacks in strength and, depending on the purpose of the usage, may present a problem such as some DNA immobilized gel particles that fall off the surface of the fiber. Therefore, the melting point of the thermoplastic resin is preferably 50° C. or higher. Especially preferred examples of the plastic include high density polyethylene and low density polyethylene. In this context, a fiber used may have a structure where the partial or entire surface of the fiber is composed of a thermoplastic resin having (in part) a relatively low melting point. For example, a composite fiber can preferably be utilized, wherein a thermoplastic resin that satisfies a melting point within the above-described range is placed on the surface of the fiber, with a plastic having a higher melting point used as a core.

The fiber on which DNA immobilized particles are bonded has a fiber diameter on the order of 0.1 μm to 3 mm, preferably 5 μm to 500 μm. It is desired that the fiber diameter should fall within this range and should be 1 or more time(s) greater, more preferably 3 or more times greater than the average particle size of the particles bonded thereon. The use of the fiber having such a fiber diameter allows the stable attachment of the particles to the surface of the fiber. The optimal relationship between a fiber diameter and a particle size differs depending on whether an object on which the particles are bonded is a single fiber substance where fibers are stretched and arranged one by one or a fiber sheet such as woven or nonwoven cloth where fibers are intertwined with each other. Especially for the fiber sheet, the optimal particle size varies according to a fiber diameter as well as the size of a void between fibers. Therefore, the optimal combination of a fiber diameter and a particle size can appropriately be determined by conducting preliminary tests. The particle size of the particles to be bonded is preferably 0.1 to 500 μm, more preferably 1 to 100 μm, as described in the discussion about the method of preparing the DNA immobilized particles. However, the particles may have a particle size exceeding this range or a particle size larger than a fiber diameter before being bonded, as long as the particles are shaped into fine particles during the process of bonding so that the resulting particles have a particle size that falls within the range or is smaller than the fiber diameter. The selection of the particles to be bonded differs depending on the place, purpose, and so on of its usage, for example, as a filter. For example, when an adsorption capacity is desired, the use of large particles is preferred because of increasing the weights of particles that can be bonded. On the other hand, when the rate of adsorption is desired, the use of small particles is preferred because of reducing the weight of particles that can be bonded but increasing the surface areas of the bonded particles. In this regard, the combination of a fiber or a fiber sheet having a small fiber diameter and DNA immobilized gel particles having a small particle size increases the surface areas of both fiber and particles. This combination also accelerates the rate of adsorption and increases an adsorption capacity to a certain degree.

The preliminary heating temperature of the DNA immobilized particles for bonding the particles onto the fiber or the fiber sheet relies on the melting point of the plastic forming the surface of the fiber and the temperature of the air stream. The preliminary heating temperature is preferably 150° C. or lower for maintaining the double helix of DNA and is 50° C. or higher, more preferably 70° C. or higher, in light of the adhesiveness of the particles to the fiber. In addition, a shorter duration of heating of these particles is more desirable in light of the stability of DNA embedded in the particles. The duration of heating may be a period of time from 1 minute to 30 minutes in light of bonding strength to the surface of the fiber. Any of methods that allow the contact or collision of the particles to be bonded with the fiber or the fiber sheet at a desired temperature may be employed for supplying the particles to the surface of the fiber. When this bonding procedure is continuously practiced, the fiber or the fiber sheet is sequentially supplied at a constant rate while the fiber or the fiber sheet is sprayed with, for example, particles heated to a given temperature together with an air stream so that they collide with each other. In the case of a fiber bundle, it is preferred that the fiber bundle should be almost evenly widened to a fixed width and this widened surface should be sprayed and supplied with the particles. Similarly, in the case of the fiber sheet, it is preferred the particles should be sprayed and supplied onto the surface of the sheet.

The temperature of the air stream through which the DNA immobilized particles are lead to collide with the surface of the fiber may be a temperature not lower than the melting point of the surface of the fiber. However, if the air stream has an exceedingly high temperature, the surface of the fiber is drastically molten, and the particles are buried into the fiber. As a result, an expected adsorption function may be impaired, or the fiber on which the particles are bonded may be broken. From this viewpoint, it is preferred that a temperature at which the particles are heated should be set to a temperature that does not exceed a temperature range of approximately 100° C. higher than the melting point of the thermoplastic resin composing the surface of the fiber for bonding. An upper limit on the temperature is 250° C. or lower, more preferably 200° C. or lower. The flow rate of the air stream relies on the thermal property of the surface of the fiber and the size and specific gravity of the particles. Therefore, any flow rate of the air stream can appropriately be determined according to the design.

In the fiber or the fiber sheet thus obtained where the DNA immobilized particles are bonded, the particles are present independently from each other on the surface of the fiber without being aggregated (in some cases, the particles come in contact with each other). For this reason, the feel and texture of the fiber and the fiber sheet are not impaired. Therefore, the fiber and the fiber sheet can be processed into a variety of shapes and can assume a form that can be used in a desired application. The fiber sheet used herein refers to nonwoven or woven cloth or a mesh-like sheet where at least the partial or entire surface of a fiber composing the fiber sheet is composed of a thermoplastic resin. For example, the fiber sheet in the form of nonwoven cloth can be utilized as a filter either directly or by sandwiching the nonwoven cloth between other nonwoven clothes having a good shape retaining property and making ridges and grooves thereon to increase a filtration area. The fiber sheet can be wrapped around a cylindrical pipe with holes made on the periphery and can also be utilized in a cartridge-style liquid filter. For example, the fiber on which DNA immobilized particles have already been bonded can be used in such a way that: the fiber can be processed into nonwoven cloth or fabric and utilized in the same way as the above-described fiber sheet in the form of nonwoven cloth; and the fiber can be formed directly into a bundle, which is then utilized with it hung and fixed in the water.

### EXAMPLES

Referring to Examples of the present application, a result of evaluating the ability to adsorb ethidium bromide, one of mutagens, will be illustrated and described hereinafter. In these Examples, the present invention will be described by quoting shapes, dimensions, numerical conditions and other particular conditions by way of illustrations for facilitating the understanding of the description. However, the present invention is not limited to these particular conditions, and variations and modifications can be made therein within the scope of the object of the present invention.

### Preparation Example 1 of DNA Immobilized Gel Particles

At first, 5 parts by weight of double-stranded DNA (average molecular weight: 6×10⁶ daltons) obtained from a salmon soft roe was dissolved in 1000 parts by weight of ion exchanged water over 1 day to yield a DNA aqueous solution. Subsequently, 20 parts by weight of commercially available alumina sol having 20% by weight of solid contents (trade name: ALUMINA SOL 520; manufactured by Nissan Chemical Industries) was added with stirring to 800 parts by weight of commercially available silica sol having 30% by weight of solid contents (trade name: “SNOWTEX CM”; manufactured by Nissan Chemical Industries). The resulting dispersion solution of DNA was then dried at 50° C. for 24 hours to yield a DNA immobilized porous oxide gel containing approximately 2% by weight of DNA. This dried gel was pulverized with a ball mill to give a DNA immobilized porous particles according to Preparation Example 1 having a particle size of approximately 20 μm.

### Preparation Example 2 of DNA Immobilized Gel Particles

At first, 100 parts by weight of H₂NC₂H₄NHC₃H₆Si(OC₂H₅)₃ was added to 1000 parts by weight of ion exchanged water and reacted for 5 days. From the resulting mixture, approximately 900 parts by weight of a dispersion medium was removed by distillation at 60° C. with an evaporator. Then, 200 parts by weight of ion exchanged water was added to the mixture to yield approximately 400 parts by weight of an aqueous solution of siloxane with a basic functional moiety. Subsequently, 5 parts by weight of double-stranded DNA (average molecular weight: 6×10⁶ daltons) obtained from a salmon soft roe was dissolved in 1000 parts by weight of ion exchanged water over 1 day to yield a DNA aqueous solution. Then, 65 parts by weight of the solution of siloxane with a basic functional moiety was added to 850 parts by weight of the commercially available silica sol described above and stirred for approximately 15 minutes. The resulting dispersion solution of a colloid was mixed with the DNA aqueous solution to yield a dispersion solution of the DNA and the colloid, which was in turn subjected to a spray drying method using air at 150° C. to give DNA immobilized porous particles according to Preparation Example 2 having a particle size of approximately 50 μm and containing approximately 1.8% by weight of DNA.

Preparation of DNA Supporting Fiber

In this Example, a polyethylene fiber having a fiber diameter of approximately 20 μm (melting point: approximately 135° C.) was used as a fiber for supporting the DNA immobilized gel particles. At first, 100 fibers were wrapped in a bundle around a roll. This fiber bundle was winded off the roll and then uniformly widened into a width of approximately 50 mm. The technique shown in the above Patent Document 3 was applied to the widened surface of this fiber bundle winded off. Namely, the above-described oxide particles were heated in advance to varying preliminary heating temperatures and stored in a hopper. The duration of storage in the hopper was standardized at 3 minutes for each temperature. These particles maintained at given temperatures were then supplied in a predetermined amount by means such as an ejector and brought into contact with the surface of the fiber through an air stream standardized at a temperature condition of 160° C., to bond the particles onto the surface of the fiber. After a reasonable period of time, the fiber on which the particles had been bonded was cooled to around room temperature and reeled on a roll, with excessive powders blown off with an air gun. The resulting fiber was used as a sample for evaluation.

(Preparation of DNA Supporting Fiber Sheet)

In this Example, nonwoven cloth (surface density: approximately 50 g/m²) produced by paper making in a wet process from core-in-sheath composite fibers composed of polyethylene having a fiber diameter of approximately 10 μm (melting point: approximately 135° C.) that served as a sheath and polypropylene (melting point: approximately 160° C.) that served as a core was used as a fiber sheet. The same technique as in the DNA supporting fiber was applied to the 50-mm-wide nonwoven cloth. The particles were heated at varying preliminary heating temperatures and stored in a hopper. After the particles were bonded onto the nonwoven cloth, from which excessive powders were removed to give a sample for evaluation.

### Example 1

The DNA supporting fiber on which the DNA immobilized gel particles according to the above Preparation Example 1 (preliminary heating temperature: 100° C.) were bonded was used as a sample for evaluation according to Example 1. A 10-m-long fiber bundle was cut out of the fiber on which the particles had been bonded. When the fiber bundle was weighed, its weight was increased from 0.35 g to 0.52 g.

### Example 2

The DNA immobilized gel particles according to the above Preparation Example 1 (preliminary heating temperature: 70° C.) was bonded onto the nonwoven cloth used as a substrate for a DNA supporting fiber sheet to give a sample for evaluation according to Example 2. The obtained nonwoven cloth sample was rendered whitish because of supporting the DNA immobilized gel particles, as compared with the nonwoven cloth before supporting the particles. A 40-cm² piece was cut out of the resulting DNA supporting nonwoven cloth. A weight gain was measured, and the amount of DNA supported thereon was shown in the table.

### Example 3

A sample for evaluation according to Example 3 was obtained in the same way as Example 2 except that a preliminary heating temperature was set to 100° C. The obtained nonwoven cloth was visually similar to the nonwoven cloth of Example 2. A 40-cm² piece was cut out of the resulting DNA supporting nonwoven cloth. A weight gain was measured, and the amount of DNA supported thereon was shown in the table.

### Example 4

A sample for evaluation according to Example 4 was obtained in the same way as Example 2 except that a preliminary heating temperature was set to 150° C. The nonwoven cloth obtained in this Example was turned white more clearly than those in Examples 1 and 2. A 40-cm² piece was cut out of the resulting DNA supporting nonwoven cloth. A weight gain was measured, and the amount of DNA supported thereon was shown in the table.

### Example 5

A sample for evaluation according to Example 5 was obtained in the same way as Example 2 except that a preliminary heating temperature was set to 100° C. and the particles according to the above Preparation Example 2 were used as DNA immobilized gel particles. The obtained nonwoven cloth was visually similar to the nonwoven cloth of Example 2. A 40-cm² piece was cut out of the resulting DNA supporting nonwoven cloth. A weight gain was measured, and the amount of DNA supported thereon was shown in the table.

The particles on the samples for evaluation obtained in Examples 1 to 5 did not easily fall off the samples by touching with hands.

### Comparative Example

A powder (0.145 g) of the DNA immobilized oxide particles according to Preparation Example 2 was directly used in evaluation.

Evaluation for Adsorption of Ethidium Bromide

Each of the samples for evaluation according to Examples and Comparative Example thus obtained evaluated for the ability to adsorb ethidium bromide, one of mutagens, by an approach described below. At first, a test solution was prepared by dissolving ethidium bromide at 57 ppm in deionized water. Each of the samples for evaluation was immersed without stirring in the test solution at room temperature for 7 days. The absorbance of each test solution at 470 nm was measured and evaluated as the amount of ethidium bromide adsorbed in each of the samples for evaluation. The absorbance I₀ of ethidium bromide at a concentration before adsorption was used to calculate the adsorption rate Iₛ of ethidium bromide (hereinafter, referred to as the EB adsorption rate) from the formula Iₛ=100×(I₀−I)/I₀ by use of the absorbance I of the solution measured after adsorption. The DNA supporting fiber and nonwoven cloth that had adsorbed ethidium bromide were irradiated with a UV lamp having a wavelength of 366 nm to observe an intercalation property under conditions of a dark room.

Each of the samples for evaluation obtained in Examples 1 to 5 was evaluated for the ability to adsorb ethidium bromide as described above. A result of the evaluation in addition to various conditions such as the weight of the fiber or the nonwoven cloth used in the evaluation is shown in Table 1. In Comparative Example, an ethidium bromide solution was directly added without stirring to 0.145 g of the powder of Preparation Example 2. After 7 days, the EB adsorption rate measured was 70%. As can be seen from this Table 1, it could be confirmed that each of the samples for evaluation of Examples 1 to 5 exhibited a relatively high value as compared with the powder of Comparative Example and rapidly expressed an adsorption property. In addition, in the investigation of the intercalation property with a UV lamp, strong fluorescence was observed in the samples of all Examples. Therefore, the function of intercalation into the double helix of DNA could be confirmed to be maintained. When the surface of the nonwoven cloth obtained in each Example is observed with an electron microscope, the particles were broken into small pieces having a particle size that was smaller than the initial particle size and was about a fraction of the fiber diameter.

TABLE 1

| | Density of | | | | | Preliminary | | |
|---|---|---|---|---|---|---|---|---|
| | nonwoven cloth | | Amount of DNA | Amount | Amount | heating | EB | Observation of |
| | (cm²) or weight | | immobilized | of DNA | of EB | temperature | adsorption | intercalation |
| | of fiber (g) | | particles (g) | (mg) | solution (g) | (° C.) | rate (%) | by UV irradiation |
| Ex. 1 | 0.52 | g | 0.17 | 0.34 | 1.2 | 100 | 96 | Strong fluorescence |
| Ex. 2 | 40 | cm² | 0.106 | 2.12 | 8.8 | 70 | 94 | Strong fluorescence |
| Ex. 3 | 40 | cm² | 0.108 | 2.16 | 8.8 | 100 | 94 | Strong fluorescence |
| Ex. 4 | 40 | cm² | 0.110 | 2.2 | 8.8 | 150 | 95 | Strong fluorescence |
| Ex. 5 | 40 | cm² | 0.102 | 1.84 | 8.1 | 100 | 93 | Strong fluorescence |

| | | | | | | |
|---|---|---|---|---|---|---|
| Com. Ex. | — | 0.145 | 2.66 | 8.8 | — | 70 |

The present Examples and Comparative Example have shown that, when the DNA immobilized gel particles of the present invention used as DNA immobilized particles were supported onto the fiber and the fiber sheet by the supporting method claimed in the present application, DNA susceptible to heat can stably be supported thereon without impairing the function of intercalation of mutagens, while the fine pores of the porous oxide particles are maintained and the adsorption property of DNA is quickly expressed.

The present invention is not limited to the above embodiments and various changes and modifications can be made within the spirit and scope of the present invention. Therefore to apprise the public of the scope of the present invention, the following claims are made.

This application claims priority from Japanese Patent Application No. 2004-342888 filed on Nov. 26, 2004, which is hereby incorporated by reference herein.

What is claimed is:

1. A DNA supporting fiber having a surface to which DNA immobilized particles are bonded, characterized in that the DNA immobilized particles are particles where DNA is immobilized in a porous matrix.

2. The DNA supporting fiber according to claim 1, wherein the porous matrix contains an inorganic oxide.

3. The DNA supporting fiber according to claim 2, wherein the inorganic oxide is capable of forming a colloid, and the particles are obtained by gelating a colloid of the inorganic oxide from a colloidal solution containing the colloid and DNA to be immobilized.

4. The DNA supporting fiber according to claim 3, wherein the colloid of the inorganic oxide is a silica colloid.

5. The DNA supporting fiber according to claim 3, wherein the colloid of the inorganic oxide is a mixture of a silica colloid and a colloid of a trivalent or tetravalent metal oxide.

6. The DNA supporting fiber according to claim 3, wherein the colloidal solution contains a polymer with a basic functional moiety.

7. The DNA supporting fiber according to claim 6, wherein the polymer is polysiloxane with a basic functional moiety.

8. The DNA supporting fiber according to claim 1, wherein at least the partial or entire surface of the fiber is composed of a thermoplastic resin.

9. A DNA supporting sheet comprising a DNA supporting fiber according to claim 1.

10. A method of producing a DNA supporting fiber having a surface to which DNA immobilized particles are bonded, characterized by comprising the step of heat sealing DNA immobilized particles where DNA is immobilized in a porous matrix to the surface including a thermoplastic resin of a fiber by supplying the DNA immobilized particles to the surface of the fiber under heating.

11. The method of producing a DNA supporting fiber according to claim 10, wherein the DNA immobilized particles are heat sealed to the surface of the fiber by bringing the DNA immobilized particles into contact with the surface of the fiber at a temperature not lower than a melting point of the thermoplastic resin forming the surface of the fiber.

12. The method of producing a DNA supporting fiber according to claim 11, wherein the DNA immobilized particles are brought into contact with the surface of the fiber with an air stream having the DNA immobilized particles dispersed therein has a temperature not lower than a melting point of the thermoplastic resin.

13. The method of producing a DNA supporting fiber according to claim 10, wherein the porous matrix contains an inorganic oxide.

14. The method of producing a DNA supporting fiber according to claim 13, wherein the inorganic oxide is capable of forming a colloid, and the particles are obtained by gelating a colloid of the inorganic oxide from a colloidal solution containing the colloid and DNA to be immobilized.

15. The method of producing a DNA supporting fiber according to claim 14, wherein the colloid of the inorganic oxide is a silica colloid.

16. The method of producing a DNA supporting fiber according to claim 14, wherein the colloid of the inorganic oxide is a mixture of a silica colloid and a colloid of a trivalent or tetravalent metal oxide.

17. The method of producing a DNA supporting fiber according to claim 14, wherein the colloidal solution contains a polymer with a basic functional moiety.

18. The method of producing a DNA supporting fiber according to claim 17, wherein the polymer is polysiloxane with a basic functional moiety.

19. The method of producing a DNA supporting fiber according to claim 10, wherein the DNA immobilized particles are brought into contact with the surface of the fiber, with the DNA immobilized particles heated at a preliminary heating temperature of 50° C. to 150° C.
